(19) United States
(12) Patent Application Publication
(10) Pub. No.: US 2001/0023317 A1
(43) Pub. Date: Sep. 20, 2001

(54) Needle with electrodes for electroporation-mediated gene therapy

(76) Inventor: Albert Wai-Kit Chan, Flushing, NY (US)

Correspondence Address: Albert Wai-Kit Chan, 59-42 Parsons Blvd., Flushing, NY, 11365-1433, US

(21) Appl. No.: 09/791,937

(22) Filed: Feb. 23, 2001

Related U.S. Application Data
(60) Provisional application No. 60/184,471, filed on Feb. 23, 2000.

(51) Int. Cl. A61B 5/04; A61M 5/32

(52) U.S. Cl. 600/393; 604/272

(57) ABSTRACT

This invention provides a needle with electrodes for delivery of nucleic acid or therapeutic agents for medical use in gene therapy. This invention also provides an apparatus which has one injection needle in the middle and two electrode needles on each side of the injection needle. Finally, this invention provides methods for delivering the nucleic acid using said needle or apparatus.

This application claims benefit of U.S. Ser. No. 60/184,471, filed Feb. 23, 2000, the content of which is incorporated by reference into this application.

### FIELD OF THE INVENTION

This invention relates to an intramuscular entry needle with electrodes (IENE) for electroporation-mediated intramuscular gene therapy. This IENE in one application facilitates the uptake of naked nucleic acid into the interior of muscular cells.

### BACKGROUND

Gene delivery into living tissues has significant implications in gene therapy and various clinical applications. Among the nonviral techniques for gene transfer in vivo, the direct injection of plasmid DNA into muscle is simple, inexpensive, and safe. The postmitotic nature and longevity of myofibers permits the stable expression of transfected genes, although the transfected DNA does not usually undergo chromosomal integration. Delivery of heterologous genes into skeletal muscles has been shown to provide sustained production of proteins in the circulation. Intramuscular injection of an interleukin-5 (IL-5) expression plasmid results in IL-5 production in muscle at a level sufficient to induce marked proliferation of eosinophils in the bone marrow and eosinophil infiltration of various organs. A single intramuscular injection of an erythropoietin expression plasmid into adult mice produced physiologic elevations in serum erythropoietin levels and increased hematocrits. Most recently, administration of the secreted form of angiogenesis inhibitor endostatin results in sustained expression and secretion of endostatin into the blood circulation and a systemic inhibition of tumor growth and metastases. These results suggest that intramuscular DNA injection is a useful method of systemic delivering cytokines, growth regulators, and other serum proteins. This method, however, has not been extended to the application to human gene therapy.

One of the major obstacles by intramuscular gene transfer is the relatively low expression levels of the transfected gene. Conditions that affect the efficiency of gene transfer by intramuscular DNA injection have been analyzed. Among several favorable factors including injection of expression plasmids into regenerating muscle and formulation of expression plasmids with synthetic polymer, electroporation has generated some excitements on improvements of the gene transfer efficiency by intramuscular DNA injection. Electroporation has been widely used to introduce DNA into various types of cells in vitro. Gene transfer by electroporation in vivo has been effective for introducing DNA into mouse skin, chick embryos, rat liver, and murine melanoma. Aihara H, et al., has recently examined the efficiency of IL-5 gene transfer into muscle by electroporation in vivo. Electroporation after plasmid injection increased serum levels of IL-5 more than 10-fold compared with that of simple intramuscular plasmid injection.

The current invention is a new design of nucleic acid delivery needle apparatus, which will deliver the electroporation to the same intramuscular area where plasmid nucleic acid is injected.

### BRIEF DESCRIPTION OF THE DRAWINGS

FIG. 1 shows the presently used injection needle.

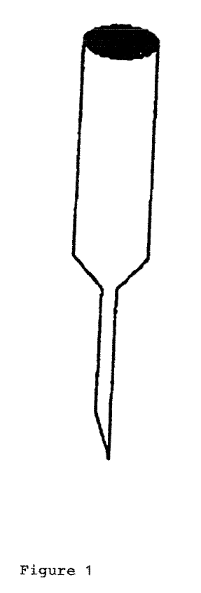

FIG. 2 shows the instantly claimed invention wherein there are three entry needles fixed on a metal frame (C), which combines a injection needle at the middle (A) and a pair of electrode needles (B). All three needles have same vertical depth (line H), which will deliver both nucleic acid and electroporation into the same layer of muscle cells. At the end of the electrode needles, there is a pair of plugs (D), which connects with electronic wire (G) on the electroporation machine. The three needles unit is mechanically supported by a triangle structure, which is consist of metal frame C, frame E, and the needle hub (F) in the middle of the triangle. The angle between frames C and E is 45° C.

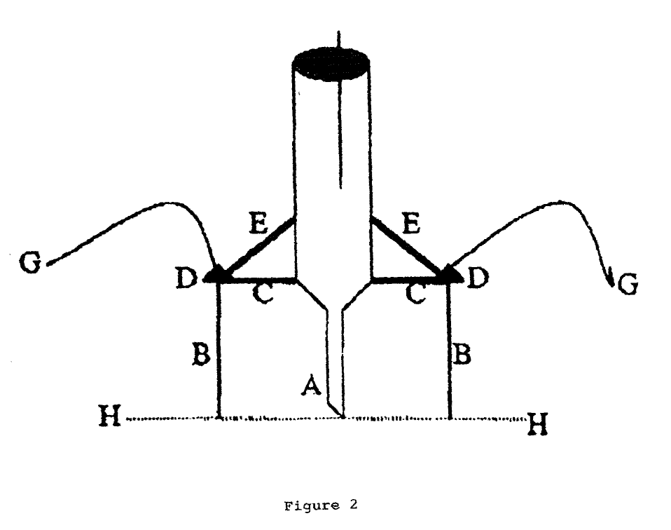

### DETAILED DESCRIPTION OF THE INVENTION

As it can be easily appreciated by an ordinary skilled artisan, the needle disclosed is useful for gene delivery via various routes which includes, but is not limited to intramuscular delivery.

Operation of a electroporation-mediated intramuscular gene delivery

1. Insert the needle into skeletal muscle.

2. Inject the nucleic acid.

3. For electroporation, a pair of electrode needles was inserted into the muscle with a 5 mm gap within the nucleic acid injection sites and same depth into the muscle as nucleic acid delivered.

4. Electric pulses were delivered using an electric pulse generator.

In an embodiment, nucleic acid is DNA. In a further embodiment the DNA is a plasmid DNA. One of the most important technical aspects in electroporation-mediated intramuscular gene delivery is to precisely deliver the electroporation to the same area where the gene is injected. With the current operation procedure, one needs to inject nucleic acid e.g. DNA plasmid, and then insert a pair of electrodes. It is difficult to insert the electrodes within the same intramuscular site and same intramuscular depth as the DNA injected. If the electroporation is not delivered into the same intramuscular area, the nucleic acid previously injected will not be efficiently introduced into the muscle cells.

With the new design of nucleic acid delivery needle apparatus, the nucleic acid delivery needle and a pair of electrodes are attached on the same applicator, which will allow delivery of both nucleic acid and electroporation to the same intramuscular area.

To prevent the generation of shortcut between two electrode needles and the DNA delivery middle needle, the middle needle has to be absent in the muscle. Therefore after the delivery of the DNA plasmid into the muscle cells, the DNA delivery middle needle will be mechanically withdrew, which left only two electrode needles in the muscle.

What is claimed is:

1. An intramuscular entry needle with electrodes for delivery of nucleic acid or therapeutic agents for medical use in gene therapy.

2. The needle of claim 1, wherein the nucleic acid is DNA.

3. The needle of claim 1, wherein the nucleic acid is delivered intramuscularly.

4. An apparatus which has one injection needle in the middle and two electrode needles on each side of the injection needle.

5. An apparatus of claim 4 comprising an injection needle, which can be withdrew from the muscle after injection of DNA.

6. An apparatus of claim 4 comprising two electrode needles, which can deliver electroporation after the withdrawing the injection needle.

7. An apparatus of claim 4 comprising two electric plugs at the end of electrode needles.

8. An apparatus of claim 4 comprising two electrode needles, which have the same vertical depth as the injection needle.

9. An apparatus of claim 4 comprising two electrode needles, which can be designed as 0.75, 1, and 1.5 cm apart.

10. An apparatus of claim 4 which has three needles fixed on the metal frame which is mechanically supported by a triangle structure with angles of 45° C.

11. A method for delivering the nucleic acid using the needle of claim 1.

12. A method for delivering the nucleic acid using the apparatus of claim 4.
